SEQUENCE LISTING (7 sequences)

SEQ ID NO: 1 - DNA, 35 bp, Artificial Sequence
  Description of Artificial Sequence Synthetic primer
gcggatccgccgtcatgacaaaggaaaatctctgg

SEQ ID NO: 2 - DNA, 57 bp, Artificial Sequence
  Description of Artificial Sequence Synthetic primer
cggaattctcacagatcctcttctgagatgagtttttgttcatgctgcgtgcgactg

SEQ ID NO: 3 - DNA, 30 bp, Artificial Sequence
  Description of Artificial Sequence Synthetic primer
cctctagagccaccatgagaggagacagca

SEQ ID NO: 4 - DNA, 31 bp, Artificial Sequence
  Description of Artificial Sequence Synthetic primer
cgaattcgaagcccttatcatttgctgtacc

SEQ ID NO: 5 - protein, 488 aa, Homo sapiens
MVAERSPARSPGSWLFPGLWLLVLSGPGGLLRAQEQPSCRRAFDLYFVLDKSGSVANNWIEIYNFVQQLAERFVSPEMRLSFIVFSSQATIILPLTGDRGKISKGLEDLKRVSPVGETYIHEGLKLANEQIQKAGGLKTSSIIIALTDGKLDGLVPSYAEKEAKISRSLGASVYCVGVLDFEQAQLERIADSKEQVFPVKGGFQALKGIINSILAQSCTEILELQPSSVCVGEEFQIVLSGRGFMLGSRNGSVLCTYTVNETYTTSVKPVSVQLNSMLCPAPILNKAGETLDVSVSFNGGKSVISGSLIVTATECSNGIAAIIVILVLLLLLGIGLMWWFWPLCCKVVIKDPPPPPAPAPKEEEEEPLPTKKWPTVDASYYGGRGVGGIKRMEVRWGDKGSTEEGARLEKAKNAVVKIPEETEEPIRPRPPRPKPTHQPPQTKWYTPIKGRLDALWALLRRQYDRVSLMRPQEGDEGRCINFSRVPSQ

SEQ ID NO: 6 - protein, 478 aa, Danio rerio
MTKENLWSVATTATLFFCLCFSSFKAETPSCHGAYDLYFVLDRSGSVSTDWSEIYDFVKNLTERFVSPNLRVSFIVFSSRAEIVLPLTGDRSEINKGLKTLSEVNPAGETYMHEGIKLATEQMKKEPKKSSSIIVALTDGKLETYIHQLTIDEADSARKYGARVYCVGVKDFDEEQLADVADSKEQVFPVKGGFQALKGIVNSILKQSCTEILTVEPSSVCVNQSFDIVLRGNGFAVGRQTEGVICSFIVDGVTYKQKPTKVKIDYILCPAPVLYTVGQQMEVLISLNSGTSYITSAFIITASSCSDGTVVAIVFLVLFLLLALALMWWFWPLCCTVVIKDPPPQRPPPPPPKLEPDPEPKKKWPTVDASYYGGRGAGGIKRMEVRWGEKGSTEEGARLEMAKNAVVSIQEESEEPMVKKPRAPAQTCHQSESKWYTPIRGRLDALWALLRRQYDRVSVMRPTSADKGRCMNFSRTQH

SEQ ID NO: 7 - protein, 476 aa, Danio rerio
MRGDSNHAITANWVLQLFCLSLLTPFGTGEDGSCHGAFDLYFVLDRSGSVSDNWLEIYGFVEQLMVNLKYPKMRVSFIVFSSSAEIILPLTGDRVDIDSGLQQLSKIRPAGDTYMHEGLKKAIEQMTSQGARASSIIIALTDGKLEVFMNELAIKEADLARQYGARVYCVGVKDFDANQLAEIADNKDQVFPVVDGFQALKNIVNSILQKSCVEVFKLEPSSICVNESFNIVLRGRGFTQGRTSESVVCTLSVNHQDPYNERPSIVKDNYVLCPAPVLTEVGQSIDVLISLNNGQSFISTPITIYATTCSDGMVAVIVILVLLVLVGLILLWWFWPLCCTVVIRDPPPSRPPPPRPIPPEIDPFPKKKWPTVDASYYGGRGPGGITRMEVRWGEKGSTEEGTRLAKAKNAVVKMPEEEFEEPVITPPPRPPPTYQAPVPDKWYTPIKGRFDAVAALLRRQYDRVAIMRPTANDKGF

(19) United States
(12) Patent Application Publication
(10) Pub. No.: US 2009/0280130 A1
(43) Pub. Date: Nov. 12, 2009

(54) METHOD FOR THE TREATMENT OF ANTHRAX TOXICITY

(75) Inventors: Joanne Chan, Medford, MA (US); Robert E. Bolcome, III, Allston, MA (US)

Correspondence Address: DAVID S. RESNICK, NIXON PEABODY LLP, 100 SUMMER STREET, BOSTON, MA, 02110-2131, US

(73) Assignee: CHILDREN'S MEDICAL CENTER CORPORATION, Boston, MA (US)

(21) Appl. No.: 12/304,495

(22) PCT Filed: Jun. 13, 2007
(86) PCT No.: PCT/US07/13813

§ 371 Date: Dec. 12, 2008

Related U.S. Application Data
(60) Provisional application No. 60/813,755, filed on Jun. 14, 2006.

(51) Int. Cl. A61K 39/395; A61K 31/545

(52) U.S. Cl. 424/145.1; 514/192

(57) ABSTRACT

Bacillus anthracis is a spore-forming Gram positive bacterium that is the causative agent of anthrax infection. Vascular leakage and pleural effusions are hallmarks of the fulminant phase of human anthrax disease following infection. The present invention provides a method of halting, treating, and preventing the rapid toxic effects of human anthrax disease by blocking the VEGF pathway with chemical inhibitors of the VEGFR signaling pathway. The invention is also applicable as an anti-anthrax therapeutic in bio-warfare defense.

### CROSS REFERENCE TO RELATED APPLICATION

The application claims benefit under 35 U.S.C. § 119(e) of the U.S. provisional application No. 60/813,755 filed Jun. 14, 2006, the content of which is herein incorporated by reference in its entirety.

Applicants assert that the paper copy of the Sequence Listing is identical to the Sequence Listing in computer readable form found on the accompanying computer disk. Applicants incorporate the contents of the sequence listing by reference in its entirety.

### BACKGROUND OF THE INVENTION

Bacillus anthracis is a spore-forming Gram positive bacterium that is the causative agent of anthrax infection. Generally, the spores can enter a subject by oral ingestion, through the skin, or by inhalation. The spores are phagocytized and travel to regional lymph nodes where they germinate and release three proteins (toxins) that are the causative agents of the symptoms of the infection. While antibiotics such as ciprofloxacin, penicillins, and tetracyclines may be effective in reducing the bacterial infection itself, once the proteins are released, reduction of the infection itself does not significantly arrest the course of the disease. In addition, in light of the attractiveness of anthrax as a biological weapon, modification of wildtype B. anthracis to provide antibiotic resistance is a distinct possibility.

Therefore, it is important to provide a means to treat anthrax which is independent of antibiotic resistance, and which will be effective even after the toxins have been released from an infection that has not been prevented or treated sufficiently promptly.

### SUMMARY OF THE INVENTION

Embodiments of the invention provide a method for treating anthrax disease in a subject in need thereof, comprising administering an effective amount of a VEGF inhibitor and a pharmaceutically acceptable carrier.

In one embodiment, the method of treating anthrax disease comprises the VEGF inhibitor that is selected from the group consisting of bevacizumab, VEGF Trap (chimeric VEGF-binding proteins from Regeneron/Aventis), CP-547,632, AG13736, AG28262, SU5416, SU11248, SU6668, ZD-6474, ZD4190, CEP-7055, PKC 412, AEE788, AZD-2171, sorafenib, vatalanib, pegaptanib octasodium, IM862, DC101, angiozyme, Sima-027, caplostatin, and neovastat.

In one embodiment, the VEGF inhibitor used in treating anthrax disease is administered by pulmonary administration. In another embodiment, the VEGF inhibitor is administered by parenteral administration. In yet another embodiment, the VEGF inhibitor used in treating anthrax disease is administered by oral administration.

In one embodiment, the method of treating anthrax disease is further comprised of administering antibiotics, wherein the antibiotics are fluoroquinolones, tetracyclines and/or penicillins.

Embodiments of the invention also provide a use of a VEGF inhibitor and a pharmaceutically acceptable carrier for the treatment of anthrax disease in a subject in need thereof.

Embodiments of the invention also provide a use of a VEGF inhibitor in the manufacture of a medicament for the treatment of anthrax disease in a subject in need thereof.

### BRIEF DESCRIPTION OF THE DRAWINGS

FIG. 1 shows blood circulation in a zebrafish at 48 hours post fertilization (hpf). Abbreviations are: DA, dorsal aorta; PCV, posterior cardinal vein; CCV, common cardinal vein. The arrow indicates the heart. The grey arrow indicates site of injection.

FIG. 2. LeTx effects in zebrafish embryos. (A) Diagram of zebrafish embryo at 48 hpf, the time point of treatments, showing functional vasculature. The grey needle indicates injection site. B-D, at 48 hpf, zebrafish embryos were injected with an inert phenol red dye (B), LeTx (C), or treated for 6 h with 2.5 μM CI-1040, then washed out (D). Images were taken at 68 hpf. Scale bar for A-D=250 μm. Abbreviations are: DA, dorsal aorta; PCV, posterior cardinal vein; CCV, common cardinal vein. Arrow indicates heart and pericardial edema in C and D. Scale bar for =80 μm.

FIG. 3. Endothelial leakage in the zebrafish heart indicated by sized fluorescent microspheres. A and B are cartoons depicting wildtype and toxin injected zebrafish hearts respectively, showing the fluid leakage and accumulation in the endocardium and myocardium in the treated zebrafish heart. Abbreviations are: E, endocardium; M, myocardium; A, atrium; V, ventricle.

FIG. 4. Validation of anthrax toxin internalization using known pathway inhibitors. Comparison of inhibitor efficacy conducted in a single representative experiment, conditions for each lane are indicated. Toxin phenotypes (severe, mild or wildtype appearance) were converted to percentages. Toxin phenotype is characterized by ISV collapse (see FIG. 8A) and pericardial edema. Severe phenotype has a complete loss of blood flow, whereas mild phenotype features limited circulation in the major axial vessels. Embryos were injected with protein inhibitors as indicated at 48 hpf and were scored at 20 hpi (hours post injection). LeTx was used at 25 fMoles PA and 37 fMoles LF. Treatments in each lane were as follows: 1. LeTx, n=25; 2. LeTx with 6 pMoles LFN (low), n=28 (P=0.912); 3. LeTx with 12 pMoles LFN (high), n=28 (P=0.007); 4. LeTx with 6 pMoles sol-CMG2 (low), n=23 (P<0.001); 5. Injection of 37 fMoles of LF alone, n=20 (P<0.001); 6. 37 fMoles LF and 12.5 fMoles PA (50%), with 12.5 fMoles PA F427A (50%), n=32 (P<0.001); 7. 37 fMoles LF and 25 fMoles PAF427A, n=30 (P<0.001); 8. 25 fMoles PA, n=31 (P<0.001); 9. 25 fMoles PAF427A, n=30 (P<0.001); 10. Uninjected control embryos, n=30 (P<0.001). Statistics were completed using the Chi Square Test.

FIG. 5. Chemical VEGFR inhibition attenuates LeTx effects. Graph of kinase inhibitor effects on LeTx phenotype conducted in a single representative experiment of three or more repeated experiments for each inhibitor. This chart graphically depicts the data reported in Table 3. ZM323881 and SU11652, selective inhibitors against VEGFR, demonstrated protection against LeTx effects. All inhibitors were used at 1.5 μM, except for ZM323881 (1 μM).

FIG. 6. VEGFR inhibition reduces LeTx induced endothelial permeability in mouse skin microvasculature. LeTx induced Evans Blue Dye leakage, representative of 6 mice. For this experiment, all mice were injected with the four depicted treatment conditions in the same orientation as shown. The spread of Evans Blue Dye indicated leakage due to enhanced endothelial permeability and the spread is measured. Data represents the average (n=6) of the observed dye leakage for this experiment. Error bars denote standard error. Both ZM323881 and SU11652 attenuated LeTx induced vascular leakage significantly (ZM323881, P=0.001; SU11652, P<0.001; LF, P<0.001). Statistics were completed using the Holm-Sidak method.

FIG. 7. Zebrafish CMG2 orthologues closely resemble human CMG2. Alignment of human CMG2 (SEQ. ID. No. 5) with predicted sequences of the zebrafish CMG2A (SEQ. ID. No. 6) and CMG2B (SEQ. ID. No. 7) genes. Regions of identity are in solid lined boxes. Protein domains were predicted by SMART (Schultz et al., 1998). The putative signal peptide region at the amino terminus ˜25-30 amino acids are in italics and non-bold characters; the von Willebrand A (vWA) domains are underline with solid lines; and the transmembrane domain are indicated by non-bold characters respectively. Residues of the metal ion dependent adhesion site (MIDAS) motif are highlighted in broken line boxes. Conserved Tyr119 and His121 residues, which are important to PA-CMG2 binding, are underline with solid lines (Santelli et al., 2004). Cytoplasmic prolines and tyrosines that may be important in receptor signaling are highlighted by asterisks.

FIG. 8. Progression of LeTx Phenotype over time. A. Cartoon of the typical blood connections affected by LeTx: the intersegmental vessels—ISVs; DLAV: Dorsal longitudinal anastomotic vessel; and the aorta. The graph shows the phenotypic progression of a population of 30 embryos from a normal appearance, through the development of toxin phenotypes, to their end stage mild or severe phenotype.

### DESCRIPTION OF THE INVENTION

Anthrax disease begins with cold like symptoms that could be easily dismissed, then progresses rapidly into a second, fulminant phase, where pleural effusions and hemorrhagic mediastinitis have been noted in the majority of patients (Swartz, M. N., 2001). Despite effective antibiotics, symptoms can persist and death may still ensue, due to high levels of anthrax toxin proteins in the blood stream (Mourez, M., 2004; Mock, M. & Fouet, A., 2001). The vasculature is an important site for the progression of anthrax disease in humans as lung edema has been attributed to increased vascular permeability (Jernigan, J. A., et. al., 2001; Kyriacou, D. N., et. al., 2004). Pathological analysis of tissue samples indicated vascular defects and edema in non-human primates (Leendertz, F. H., et. al., 2004), as well as in two strains of mice (Moayeri, M., et. al., 2003). Although mammalian models can provide a great deal of information, the inability to examine cellular damage without sacrificing the animals limits their use to more focused studies. The zebrafish embryo is a versatile model system that can be used to dissect the entire process of toxin action. As a vertebrate organism, the conservation of developmental processes, organ systems, genes and signaling pathways has facilitated its use in forward genetics, developmental biology, and more recently, for chemical biology (Fishman, M. C., 2001; Zon, L. I. and Peterson, R. T. 2005). The fecundity of the zebrafish has also contributed to its usefulness, enabling the researcher to evaluate the consequences of pathway disruption using a large number of embryos.

We have developed a highly reproducible, novel model for anthrax toxicity in the transparent zebrafish embryo by delivery of the toxin proteins into the vasculature. Toxin induced defects include, but should not be construed to be limited to, the loss of endothelial cell function including increased permeability, leading to cardiac valve dysfunction, blood vessel collapse and rapid onset of edema, demonstrating a loss of vascular integrity, all aspects of the human disease. The specificity of toxin action is confirmed through several distinct lines of evidence: single components of the bipartite toxin do not induce toxicity; toxin effects appear to be mediated by the zebrafish anthrax toxin receptors; protein inhibitors to block toxin-receptor binding, toxin assembly, or intracellular toxin delivery all provide protection against anthrax toxicity. Since lethal toxin is known to inactivate MAPKK/MEK kinase pathways, we showed that these defects in the zebrafish could be phenocopied by the use of a small molecule MEK inhibitor. In addition, these effects can be analyzed from 1 to 24 hours after injection, using large sample numbers per experimental condition. The transparency and availability of cell type-specific transgenic lines such as the endothelial-EGFP line (Tg(fli1:EGFP)y1) (Lawson, N. and Weinstein, B., 2002) facilitate in vivo analysis of cell behavior. These assays provide rapid results that can also be widely used to study vascular receptor signaling pathways.

The present invention is based on the discovery that anti-angiogenic therapies are useful for treating anthrax disease. Anti-angiogenic therapies are particularly useful in reducing vascular permeability and thus ameliorating the anthrax-associated symptoms of lung edema and pleural effusions. In one embodiment, the invention provides a method for treating anthrax disease in a subject in need thereof, comprising administering an effective amount of a VEGF inhibitor and a pharmaceutically acceptable carrier. In another embodiment, the invention also provide methods for treating and/or preventing symptoms of anthrax toxicity in a subject in need thereof. The methods comprise administering a therapeutically effective amount of anti-angiogenic agents, preferably VEGF inhibitors.

In one embodiment of the invention, the VEGF inhibitor is selected from the group consisting of bevacizumab, VEGF Trap, CP-547,632, AG13736, AG28262, SU5416, SU11248, SU6668, ZD-6474, ZD4190, CEP-7055, PKC 412, AEE788, AZD-2171, sorafenib, vatalanib, pegaptanib octasodium, IM862, DC101, angiozyme, Sirna-027, caplostatin, and neovastat.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

A prophylactically or therapeutically effective amount means that amount necessary to attain, at least partly, the desired effect, or to delay the onset of, inhibit the progression of, prevent the reoccurrence of, or halt altogether, the onset or progression of the particular condition being treated, e.g., anthrax toxicity, e.g., anthrax-associated lung edema and pleural effusion. Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. It is preferred generally that a maximum dose be used, that is, the highest safe dose according to sound medical judgment. It will be understood by those of ordinary skill in the art; however, that a lower dose or tolerable dose may be administered for medical reasons, psychological reasons or for virtually any other reason.

The term “therapeutically effective amount” refers to an amount that is sufficient to effect a therapeutically or prophylactically significant reduction in anthrax toxicity when administered to a typical subject who is infected with anthrax or who is at risk of being infected or reinfected with anthrax disease. A therapeutically or prophylactically significant-reduction, e.g., reduction in pleural effusion, is about 30%, about 40%, about 50%, about 60%, about 70%, about 80%, about 90%, about 100%, about 125%, about 150% or more compared to a control.

As used herein, the term “medicament” refers to an agent that promotes the recovery from and/or alleviate the symptoms of anthrax disease.

The term “preventing” as used herein refers to preventing anthrax-toxicity and associated symptoms, e.g., pleural effusion, in an individual infected, but symptomless, an individual suspected to be infected or an individual susceptible for infection or re-infection. Accordingly, administration of a prophylactic agent can occur prior to the manifestation of symptoms characteristic of anthrax toxicity, such that anthrax toxicity is prevented or, alternatively, delayed in its progression. In one embodiment, the VEGF inhibitor is administered by pulmonary administration. In an alternate embodiment, the VEGF inhibitor is administered by parenteral administration. Any mode of administration of the therapeutic agents of the invention, as described herein or as known in the art, including pulmonary, nasal, oral, parenteral or enteral administration of methods of the instant invention, may be utilized for the prophylactic treatment of an infectious disease or disorder.

For use in treatment, the compounds of the invention are administered in standard protocols, for example by parenteral, such as intravenous administration. Preferably, the typical dosage level per day is about 10 mg/kg; however, this is merely a starting point as a number of factors need to be considered in determining dosage. In general, any satisfactory route of administration may be employed. In addition to intravenous, intranasal or intrapulmonary administration, which are preferred, intramuscular, intraperitoneal, or subcutaneous injection may be used. The invention compounds may also be delivered through transmucosal or transdermal routes, or may be administered by inclusion in a controlled release matrix. In addition, liposomal preparations or other particulate preparations associated with drug delivery may be employed.

Some anti-VEGF compounds may be administered orally. A typical dose regimen would include, for example, 1-4 doses per day using tablets or capsules containing approximately 500 mg of active ingredient, with 1-4 capsules or tablets being administered per dose. Optimization of dosage regimen is routine and will depend on the nature of the active ingredient, the severity of the infection, the condition of the subject, and the judgment of the practitioner. Optionally, a compound may be administered in a combination of oral, pulmonary and parenteral regimens.

“Subject”, as used herein, can refer to a mammal, e.g., a human, or to an experimental or animal or disease model, such as a mouse, rat, or rabbit. The subject can also be a non-human animal, e.g., a horse, cow, goat, or other domestic animal.

As stated above, the specific means of administration and the dosage level will be dependent on the nature of the active ingredient, the nature of the subject to be treated, the severity of the infection and/or lethal factor intoxication, the severity of the risk of infection and/or lethal factor intoxication, and the judgment of the practitioner.

Embodied in the invention are both prophylactic and therapeutic methods of treating a subject at risk of (or susceptible to) developing lethal factor toxicity as a result of anthrax exposure or toxicity, and/or to subjects exhibiting anthrax disease which include having lethal factor toxicity. As used herein, the prophylactic and therapeutic methods are defined as the application or administration of a compound or composition to a patient, or application or administration of a compound or composition to a tissue in a patient, who has an infection, a symptom of an infection or a predisposition toward an infection, with the purpose to cure, heal, alleviate, relieve, alter, remedy, ameliorate, improve or affect the infection, the symptoms of infection or the predisposition toward infection.

Inhibitors of VEGF used in the methods described herein for treating subjects for the toxic effects of anthrax infection can be used in combination with additional VEGF inhibitors and/or in combination with antibiotics or other pharmaceutically active ingredients for the amelioration of side effects or enhanced effectiveness. Antibiotics include, for example, fluoroquinolones such as ciprofloxacin, tetracyclines such as doxycycline, and penicillins. Therapeutics for the amelioration of other symptoms of anthrax and anthrax disease toxicity may also be administered in conjunction with the methods described herein.

Anti-toxin agents against the lethal toxins produced by B. anthracis can be used in combination with the methods of the inventions for treating subjects for the toxic effects of anthrax infection. An anti-toxin agent as used herein refers to any organic or inorganic molecule that binds, inhibits, prevents, impedes, stops and/or blocks the toxins from interacting with its respective cellular receptors and also inhibits, prevents, impedes, stops and/or blocks the cellular signaling events after toxin-receptor interaction. Anti-toxin agents include, but should not be construed as limited to, anti-anthrax nanosponges that work as molecular decoys to grab the toxins out of the bloodstream, high-affinity anthrax anti-toxin antibody that can successfully eliminated both anthrax bacteria and its deadly toxins in animals, human monoclonal antibody against B. anthracis PA, PAmAb, hydroxamate, (2R)-2-[(4-fluoro-3-methylphenyl)sulfonylamino]-N-hydroxy-2-(tetrahydro-2H-pyran-4-yl)acetamide, adefovir, anthrax vaccine adsorbed (AVA), and the divalent cation chelating agent, EDTA. The anti-toxins can be used in combination with VEGF inhibitors and/or in combination with antibiotics or other pharmaceutically active ingredients for the amelioration of side effects or enhanced effectiveness.

The VEGF inhibitors used with the methods described herein can be incorporated into pharmaceutical compositions suitable for the administration route. Such compositions typically comprise at least one VEGF inhibitor and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Generally, the compositions of the instant invention are introduced by any standard means, with or without stabilizers, buffers, and the like, to form a pharmaceutical composition. For use of a liposome delivery mechanism, standard protocols for formation of liposomes can be followed. The compositions of the present invention can also be formulated and used as tablets, capsules or elixirs for oral administration; sprays for inhalation; sterile solutions; suspensions for injectable administration; and the like.

Examples of routes of administration include parenteral, e.g., intravenous, intramuscular, intradermal, subcutaneous, oral (e.g., inhalation), transdermal (topical), transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

The compounds can also be prepared in the form of suppositories (e.g., with conventional suppository bases such as cocoa butter and other glycerides) or retention enemas for rectal delivery.

Systemic administration can also be topical, e.g., by transmucosal or transdermal means. Suitable formulations for topical, administration include solutions, suspensions, gels, lotions and creams as well as discrete units such as suppositories and microencapsulated suspensions. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays, suppositories or the formulations of the transdermal administrations. For transdermal administration, the active compounds are formulated into ointments, salves, gels, or creams as generally known in the art. Transmucosal drug delivery in the form of a polytetrafluoroetheylene support matrix is described in U.S. Pat. No. 5,780,045 (specifically incorporated herein by reference in its entirety).

In certain preferred embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts.

Delivery systems can include sustained release delivery systems which can provide for slow release of the active component of the invention, including sustained release gels, creams, suppositories, or capsules. In one embodiment, the active compounds are prepared with carriers that will protect the compound against rapid elimination from the body, such as a controlled release formulation, including implants and microencapsulated delivery systems. Sustained release delivery systems include, but are not limited to: (a) erosional systems in which the active component is contained within a matrix, and (b) diffusional systems in which the active component permeates at a controlled rate through a polymer. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to hepatocytes) can also be used as pharmaceutically acceptable carriers. These can be prepared according to methods known to those skilled in the art, for example, as described in U.S. Pat. No. 4,522,811 and U.S. Pat. No. 5,643,599, the entire contents of which are incorporated herein.

In another embodiment, pharmaceutical compositions may be delivered by ocularly via eyedrops.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the VEGF inhibitor in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography. In one embodiment, an animal model, the zebrafish as disclosed herein, is provided for used in testing, experimentation, and establishment of safe dosage for use in clinical trials in mammals.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an VEGF inhibitor can include a single treatment or, preferably, can include a series of treatments.

Generally, at intervals to be determined by the prophylaxis or treatment of pathogenic states, doses of active component will be from about 0.01 mg/kg per day to 1000 mg/kg per day. Small doses (0.01-1 mg) may be administered initially, followed by increasing doses up to about 1000 mg/kg per day. In the event that the response in a subject is insufficient at such doses, even higher doses (or effective higher doses by a different, more localized delivery route) may be employed to the extent patient tolerance permits. Multiple doses per day can be contemplated to achieve appropriate systemic levels of compounds.

It is understood that appropriate doses of the VEGF inhibitors depend upon a number of factors within the ken of the ordinarily skilled physician, veterinarian, or researcher. The dose(s) of the agent will vary, for example, depending upon the identity, size, and condition of the subject or sample being treated, further depending upon the route by which the composition is to be administered, if applicable, and the effect which the practitioner desires from administering siRNAs that may inhibit the VEGF pathway. The siRNAs are targeted at components of the VEGF pathways.

The pharmaceutical compositions can be included in a container, pack, or dispenser together with instructions for administration.

As the VEGF/VPF protein interacts with the VEGFRs, inhibition of either the ligand by reducing the amount that is available to interact with the receptor; or by inhibiting the receptor's intrinsic tyrosine kinase activity, blocks the function of this pathway. This pathway controls endothelial cell growth, as well as permeability, and these functions are mediated through the VEGFRs. For many anti-angiogenic treatments, the need to prune overactive vessels necessitates the use of high levels of inhibition. In one embodiment, low levels of inhibition are sufficient to reduce vascular permeability associated with anthrax-associated pleural effusion. Thus, in one embodiment, the dosage of VEGF inhibitors required treatment of anthrax toxicity is less than the dosage required for treatment of abnormal angiogenesis using the same VEGF inhibitor. That is, in one embodiment, the dosage determined for use in inhibiting angiogenesis utilizing a particular VEGF inhibitor known to the skilled artisan may be reduced in the treatment of anthrax toxicity-related pleural effusion.

As used herein the term “VEGF inhibitors” refers to any compound or agent that produce a direct effect on the signaling pathways that promote growth, proliferation and survival of a cell by inhibiting the function of the VEGF protein, including inhibiting the function of VEGF receptor proteins. The term “agent” or “compound” as used herein means any organic or inorganic molecule, including modified and unmodified nucleic acids such as antisense nucleic acids, RNAi agents such as siRNA or shRNA, peptides, peptidomimetics, receptors, ligands, and antibodies. Preferred VEGF inhibitors, include for example, AVASTIN® (bevacizumab), an anti-VEGF monoclonal antibody of Genentech, Inc. of South San Francisco, Calif., VEGF Trap (Regeneron/Aventis). Additional VEGF inhibitors include CP-547,632 (3-(4-Bromo-2,6-difluoro-benzyloxy)-5-[3-(4-pyrrolidin 1-yl-butyl)-ureido]-isothiazole-4-carboxylic acid amide hydrochloride; Pfizer Inc., NY), AG13736, AG28262 (Pfizer Inc.), SU5416, SU11248, & SU6668 (formerly Sugen Inc., now Pfizer, New York, N.Y.), ZD-6474 (AstraZeneca), ZD4190 which inhibits VEGF-R2 and —R1 (AstraZeneca), CEP-7055 (Cephalon Inc., Frazer, Pa.), PKC 412 (Novartis), AEE788 (Novartis), AZD-2171), NEXAVAR® (BAY 43-9006, sorafenib; Bayer Pharmaceuticals and Onyx Pharmaceuticals), vatalanib (also known as PTK-787, ZK-222584: Novartis & Schering: AG), MACUGEN® (pegaptanib octasodium, NX-1838, EYE-001, Pfizer Inc./Gilead/Eyetech), IM862 (glufanide disodium, Cytran Inc. of Kirkland, Wash., USA), VEGFR2-selective monoclonal antibody DC101 (ImClone Systems, Inc.), angiozyme, a synthetic ribozyme from Ribozyme (Boulder, Colo.) and Chiron (Emeryville, Calif.), Sirna-027 (an siRNA-based VEGFR1 inhibitor, Sirna Therapeutics, San Francisco, Calif.) Caplostatin, soluble ectodomains of the VEGF receptors, Neovastat ( [symbol] terna Zentaris Inc; Quebec City, Calif.), ZM323881 (CalBiochem. CA, USA) and combinations thereof. VEGF inhibitors useful in the practice of the present invention are disclosed in U.S. Pat. Nos. 6,534,524 and 6,235,764, both of which are incorporated in their entirety. Additional VEGF inhibitors are described in, for example in WO 99/24440 (published May 20, 1999), International Application PCT/IB99/00797 (filed May 3, 1999), in WO 95/21613 (published Aug. 17, 1995), WO 99/61422 (published Dec. 2, 1999), U.S. Pat. Publ. No. 20060094032 “siRNA agents targeting VEGF”, U.S. Pat. No. 6,534,524 (discloses AG13736), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), U.S. Pat. No. 6,653,308 (issued Nov. 25, 2003), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), WO 01/02369 (published Jan. 11, 2001); U.S. Provisional Application No. 60/491,771 piled Jul. 31, 2003); U.S. Provisional Application No. 60/460,695 (filed Apr. 3, 2003); and WO 03/106462A1 (published Dec. 24, 2003). Other examples of VEGF inhibitors are disclosed in International Patent Publications WO 99/62890 published Dec. 9, 1999, WO 01/95353 published Dec. 13, 2001 and WO 02/44158 published Jun. 6, 2002.

In yet another embodiment, the method of treating anthrax disease in a subject in need thereof comprising administering a VEGF inhibitor and a suitable pharmaceutical acceptable carrier is further comprise administering antibiotics, wherein the antibiotics are fluoroquinolones, tetracyclines and/or penicillins.

In yet another embodiment, the method of treating anthrax disease in a subject in need thereof comprising administering a VEGF inhibitor and a suitable pharmaceutical acceptable carrier is further comprise administering an anthrax anti-toxin agent. Such examples of anti-toxin agents include as anti-toxin antibodies, toxin-binding proteins, toxin-binding peptides, and peptide mimics (peptidomimetics) designed to block the interaction of toxin with cellular receptors.

This invention is further illustrated by the following example which should not be construed as limiting. The contents of all references cited throughout this application, as well as the figures and table are incorporated herein by reference.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

### EXAMPLE 1

### Introduction

Anthrax lethal toxin (LeTx) is the combination of two toxin protein subunits, protective antigen (PA) and lethal factor (LF). PA allows the toxin to enter cells and LF cleaves cellular targets ultimately resulting in toxin phenotypes. When administered singly, PA and LF are unable to produce discernable toxin effects.

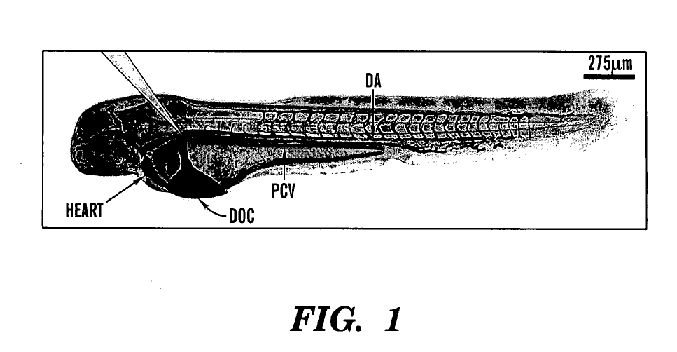

Zebrafish embryos are injected with LeTx at 48 hours post fertilization (hpf). About ˜40 nl of fluid is injected. FIG. 1 shows a diagram depicting blood circulation 48 hpf. The injection site is indicated with a grey needle. Embryos treated with drug were treated with VEGFR inhibitors from 30 minutes prior to injection and until scoring the next day. Two commercially available anti-VEGFR inhibitors were used: ZM323881 (Calbiochem catalog#676497) and SU11652 (Calbiochem catalog#572660). Data from the Calbiochem website shows that ZM323881 is a highly selective inhibitor of VEGFR2, with an IC₅₀ of less than 2 nM; whereas SU11652 can inhibit a number of kinases at high affinity: PDGFRβ (IC₅₀=3 nM), VEGFR2 (IC₅₀=27 nM), FGFR1 (IC₅₀=170 nM). These inhibitors were dissolved as stocks in DMSO at 10 mM.

Embryos are typically scored for phenotype ˜24 hours after injection. The scoring process involves recording the number of injected embryos that fit into three phenotypic categories: severe toxin phenotype, mild toxin phenotype, and wild-type appearance. This is done for each treatment condition. Severe toxin phenotype includes, but is not limited to: pericardial edema, loss of all blood circulation, collapse of intersegmental vessels along the trunk and tail, loss of heart valve function, and heart outflow tract fusion. Mild toxin phenotype essentially the same as above except that residual blood flow is retained. Wild-type appearance is scored as having no obvious effect of toxin action.

### Experiment #1

The score chart for experiment no. 1 is provided below. Condition 1 is an injection of LeTx (50 fMoles PA+74fMolesLF per embryo). This amount of LeTx is present in all conditions except that only PA is present in condition 6 (50 fMoles PA). A VEGFR inhibitor, ZM323881, is used to treat LeTx injected embryos as indicated. Individual embryos were scored visually to determine their toxin phenotypes. The proportion of embryos displaying toxin phenotypes (combination of severe and mild LeTx effects), is expressed as a percentage.

TABLE 1

| | | Severe | Mild | | Total # of | % With Toxin |
|---|---|---|---|---|---|---|
| | | Toxin | Toxin | Wild-type | Embryos | Phen (Mild + |
| | Conditions | Phen | Phen | Phen | Injected | Severe) |
| 1 | LeTx Control | 9 | 14 | 2 | 25 | 92% |
| 2 | LeTx; 1 uM ZM323881 | 1 | 8 | 10 | 19 | 47% |
| 3 | LeTx; 500 nM ZM323881 | 3 | 9 | 7 | 19 | 63% |
| 4 | LeTx; 250 nM ZM323881 | 6 | 13 | 1 | 20 | 95% |
| 5 | LeTx; 100 nM ZM323881 | 6 | 12 | 3 | 21 | 86% |
| 6 | PA | 0 | 0 | 0 | 20 | 0% |

### Experiment #2

For experiment no. 2, a higher amount of LeTx was used (˜75 fMoles PA+˜110 fMoles LF). Embryos were scored at around 28 hours post injection.

TABLE 2

| | | Severe | | | Total # of | % With Toxin |
|---|---|---|---|---|---|---|
| | | Toxin | Mild | # Wild-type | Embryos | Phen (Mild |
| | Conditions | Phen | Phen | Phen | Injected | and Severe) |
| 1 | LeTx Control | 16 | 2 | 0 | 18 | 100 |
| 2 | LeTx; 1.5 uM ZM323881 | 5 | 9 | 5 | 19 | 74 |
| 3 | LeTx; 1 uM ZM323881 | 7 | 7 | 6 | 20 | 74 |
| 4 | LeTx; 500 nM ZM323881 | 2 | 4 | 14 | 20 | 30 |
| 5 | LeTx; 250 nM ZM323881 | 13 | 5 | 1 | 19 | 95 |
| 6 | LeTx; 1.5 uM SU11652 | 9 | 7 | 4 | 20 | 80 |
| 7 | LeTx; 1 uM SU11652 | 11 | 9 | 0 | 20 | 100 |
| 8 | LeTx; 500 nM SU11652 | 11 | 8 | 0 | 19 | 100 |
| 9 | LeTx; 250 nM SU11652 | 12 | 8 | 0 | 20 | 100 |
| 10 | PA | 0 | 0 | 20 | 20 | 0 |

### Results and Discussion

In both experiments, lethal toxin (LeTx=LF and PA) is injected to show that a high percentage of embryos displaying toxin phenotypes (92% in experiment #1 and 100% in experiment #2). A negative control is used to show that injection of a single component of LeTx generated no visual defects as all injected embryos have a wild-type appearance.

In the first experiment, a range of the VEGFR inhibitor ZM323881 is used: 1 uM, 500 nM, 250 nM and 100 nM. A reduction in the proportion of embryos displaying LeTx phenotypes is seen in 3 concentrations of the inhibitor. The most striking observation is the dramatic reduction in the severe forms of the LeTx phenotype that occurred in all concentrations used. As this was the first experiment, the second experiment was designed to include an additional, structurally unrelated, VEGFR inhibitor, SU11652, and the increased the amount of LeTx used in our assay.

In the second experiment, a higher dose of LeTx ensured that 100% of LeTx injected embryos displayed toxin phenotypes. All concentrations of the selective VEGFR inhibitor, ZM323881, were able to attenuate the toxin effects. The SU11652 inhibitor was also able to protect against LeTx effects at the 1 uM concentration. At lower concentrations of these inhibitors, the proportion of severe LeTx phenotypes were drastically reduced compared with LeTx controls.

These experiments demonstrate that inhibition of the VEGFR in vivo alleviate the effects of LeTx action and treat anthrax infection.

### EXAMPLE 2

### Introduction

Bacillus anthracis releases toxin proteins into the host that cause damage in most tissues and organs, ultimately resulting in death (Firoved et al., 2005; Moayeri et al., 2003, 2004). Disruption of vascular integrity has been consistently observed in human anthrax disease (Guamer et al., 2003; Jernigan et al., 2001; Kyriacou et al., 2004), as well as in studies using mammalian models (non-human primates, rabbits, guinea pigs, rats, mice) (Beall and Dalldorf, 1966; Guarner et al., 2003; Leendertz et al., 2004; Moayeri et al., 2003; Nordberg et al., 1964; Ross, 1955; Steams-Kurosawa et al., 2006), involving infections by the bacterium or injection of toxin proteins to mimic the toxemia stage of the disease. The importance of blood vessels and endothelial cells in anthrax toxicity has been difficult to investigate due to the inability to directly observe progressive vascular changes without sacrificing the mammalian host. Therefore, a comprehensive surrogate model is needed to facilitate dissection of the host signaling pathways responsible for anthrax toxin effects.

Anthrax toxins consist of a common receptor binding component, protective antigen (PA), which can mediate cellular entry of the enzymatic components, edema factor (EF) and/or lethal factor (LF). The combination of PA with LF is known as lethal toxin (LeTx), whereas edema toxin (EdTx) refers to PA and EF. Historically, LeTx preparations were able to induce rapid death in experimental animals associated with vascular defects and pleural effusions (Beall and Dalldorf, 1966; Ross, 1955). In contrast, early reports of EdTx effects did not produce significant mortality (Beall et al., 1962). This may be due to the quality of the protein preparation, as recent reports demonstrate robust effects of EdTx including lethality in rodents (Cui et al., 2007; Firoved et al., 2005). However, the ability to induce vascular integrity loss and leakage has been consistently associated with LeTx (Cui et al., 2007; Firoved et al., 2005; Gozes et al., 2006; Moayeri et al., 2003).

Two mammalian anthrax toxin receptors (ANTXRs) are reported to bind PA: tumor endothelial marker 8 (TEM8, also known as ANTXR1) (Bradley et al., 2001) and capillary morphogenesis gene 2 (CMG2, also known as ANTXR2) (Scobie et al., 2003; Wigelsworth et al., 2004). Both receptors mediate anthrax toxin internalization and cellular delivery of LF, and are expressed in endothelial cells, as well as other cell types. During acute anthrax infections, high levels of anthrax toxin proteins in the bloodstream suggest possible interactions with endothelial ANTXRs.

To evaluate the role of LeTx action on intact blood vessels, we developed a zebrafish vascular model that permits in vivo imaging of the toxin's effects. Zebrafish embryos are transparent allowing real-time observation of blood flow, which begins from 24-26 hpf (hours post fertilization) (Isogai et al., 2001). As a vertebrate organism, zebrafish genes and signaling pathways are highly conserved with mammalian ones. The number of available embryos permits the use of large sample numbers for each assay. In our assays, LeTx is delivered into the embryonic circulation and cardiovascular function is monitored over 24 h. We found that a LeTx induced an increase in vascular permeability as the earliest observable toxin effects by 6 hpi (hours post injection). It is well-established that maintenance of normal vascular function requires tight regulation by the vascular endothelial growth factor (VEGF) signaling pathway. VEGF was first identified as the Vascular Permeability Factor (VPF) (Dvorak, 2006; Senger et al., 1983), as its ability to induce vascular leakage is unique among angiogenic growth factors. Using chemical inhibitors of VEGFR, we have demonstrated significant protection against anthrax toxicity in our zebrafish model and confirmed this finding using the Miles assay in mice (Gozes et al., 2006). By controlling vascular leakage, it may be possible to increase the window of opportunity for antibiotics and anti-toxin therapeutics to combat anthrax disease.

Experimental Procedures

Animals

All animal protocols were approved by the Institutional Animal Care and Use Committee of Children's Hospital Boston. Breeding fish were maintained at 28.5° C. on a 14 h light/10 h dark cycle. Wildtype fish used were of the AB strain. Embryos were collected by natural spawning, and raised in 10% Hank's saline at 32° C. Eight week old C3H/HeJ mice were purchased from The Jackson Laboratory (Bar Harbor, Me.) and were used within two weeks of their delivery.

Microinjection of Toxin Proteins and Inhibitors

Microinjections were carried out as described by (Weinstein et al., 1995), with modifications described by (Chan and Serluca, 2004). PA was prepared as previously described (Wigelsworth et al., 2004). LF was purchased from List Laboratories, Inc. LFDTAN was a kind gift from Stephen Juris. Combinations of LF, PA, LFNDTA, LFN, PA F427A, and sol-CMG2 were prepared immediately before, and kept at 4° C. until injection. Injected amounts are indicated in the figure legends for each experiment. Phenol red (0.05%) was added to each condition for visibility during microinjection. Volumes of 40 nl or less were delivered into the common cardinal vein of embryos anesthetized with tricaine (Sigma) at 48 hpf using a gas driven microinjector (Medical Systems Corp.). After injection, embryos were transferred into fresh medium for recovery, maintained at 32° C., and scored for toxin action at time points indicated in the text. Small molecule inhibitor treatments were conducted as described in the text and figure legends.

### Microinjection of Microspheres

Microsphere mixtures were prepared 24 h before the initiation of these experiments. 1 part each of 100 nm (diameter) blue and 500 nm red fluorescent bead suspensions (Duke Scientific Corp.) were added to 1 part 1% BSA and stored at 4° C. Control and LeTx injected embryos were microinjected with a volume of no more than 20 nl of the microsphere mixture at time points indicated in the text. Fifteen minutes following bead injection, embryos were fixed in 2% PFA for 5-10 minutes. They were then imbedded in 2% low melt agarose (Bio-Rad) on 35 mm glass bottom microwell dishes (MatTek Corp.) for confocal microscopy.

Miles Assay

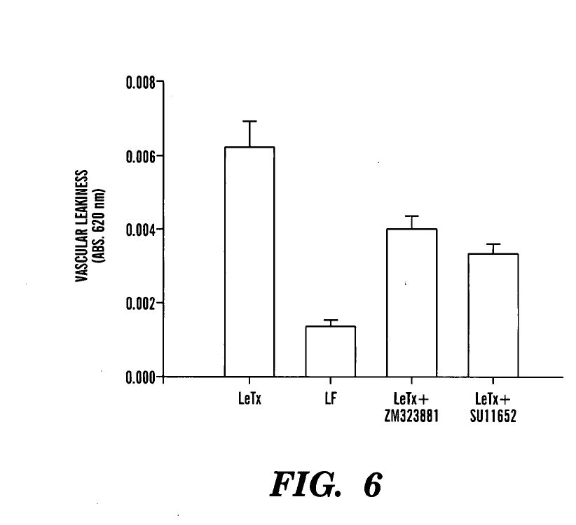

Mice were shaved 24 h prior to being injected retroorbitally with 100 μl of 1% Evans blue dye (Sigma Chemical Co., St. Louis, Mo.). After 20 min, 30 μl of LeTx+PBS, LeTx+ZM323881, LeTx+SU11652, and control samples (PA only, LF only, or phosphate-buffered saline) were injected i.d. in the left and right flanks of each animal. 60 minutes following the injection of test substances, all mice were sacrificed and equally sized (1.0-1.5 cm diameter) skin regions surrounding each i.d. injection site were removed and placed in 1 ml formamide protected from light at room temperature for five days allowing for dye extraction. The A620 of samples was read to quantify and compare leakage. Because the thickness of the skin varies along the dorsal side of the mouse, we normalized our data by dividing A620 values by the weight (ng) of each corresponding skin sample that was removed for dye extraction. Though this lowered our overall values in FIG. 6, it allowed for greater accuracy in the comparison between the leakiness produced by each treatment condition.

Isolation and Characterization of cDNA Clones

The human amino acid sequence for CMG2 (SEQ. ID. No. 5) was used to BLAST search the GenBank zebrafish EST library for translational matches. A 5′ primer (5′-GCGGATCCGCCGTCATGACAAAGGAAAATCTCTGG-3′) (SEQ. ID. No. 1) for the CMG2A transcript was designed from GenBank Accession No. CA471164. A 3′ primer (5′-CGGAATTCTCACAGATCCTCTTCTGAGATGAGTTTTTGTTCATGCTGCGTGCGAC TG-3′) (SEQ. ID. No. 2) for CMG2A incorporating a myc-tag was designed from GenBank Accession No. B1475178. A 5′ (CCTCTAGAGCCACCATGAGAGGAGACAGCA) (SEQ. ID. No. 3) and 3′ (CGAATTCGAAGCCCTTATCATTTGCTGTACC) (SEQ. ID. No. 4) primers for CMG2B were designed using GenBank Accession No. XP_{—}689332.1 and No. XM_{—}684240.1 as well as Endemble gene ENSDARG0000063011. RT-PCR was used to clone CMG2A and CMG2B into pCR II-TOPO vectors (Invitrogen). Their GenBank Accession Numbers are DQ415957 and #PENDING respectively. Nucleotide sequences were determined using the dideoxy method by the Harvard Biopolymers Facility.

Statistics

Statistical analysis was by Chi Square for zebrafish LeTx studies, the Holm-Sidak method for Miles assays, and the paired t test for LFNDTA experiments using Sigma Stat 3.0 software. P<0.05 was considered significant.

Results

LeTx Effects and Vascular Leakage in the Zebrafish

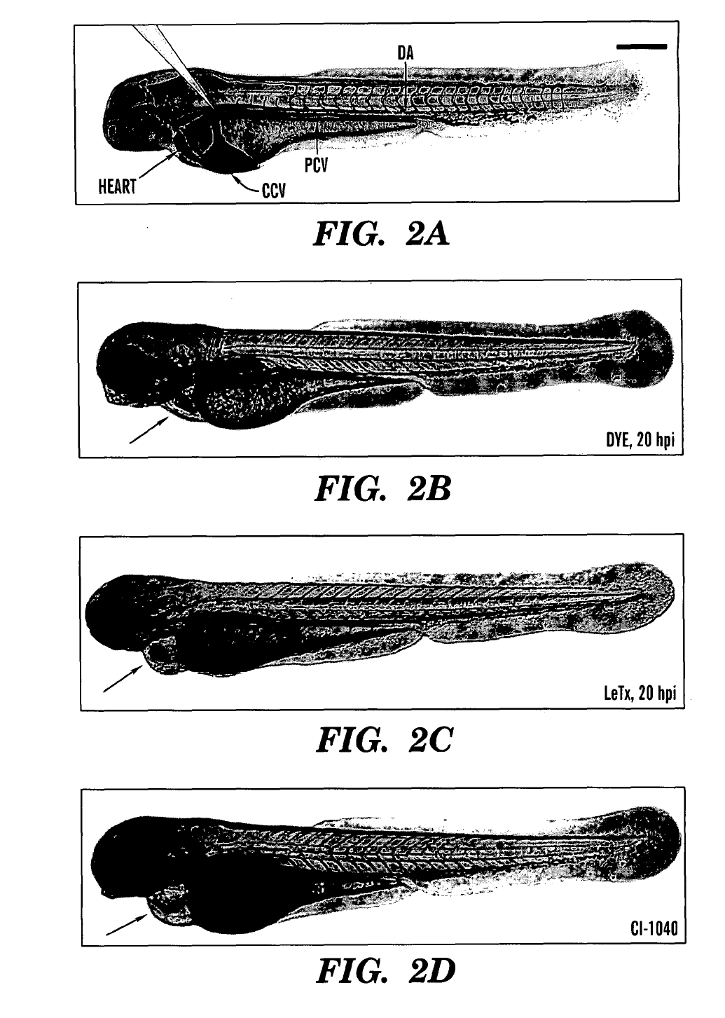

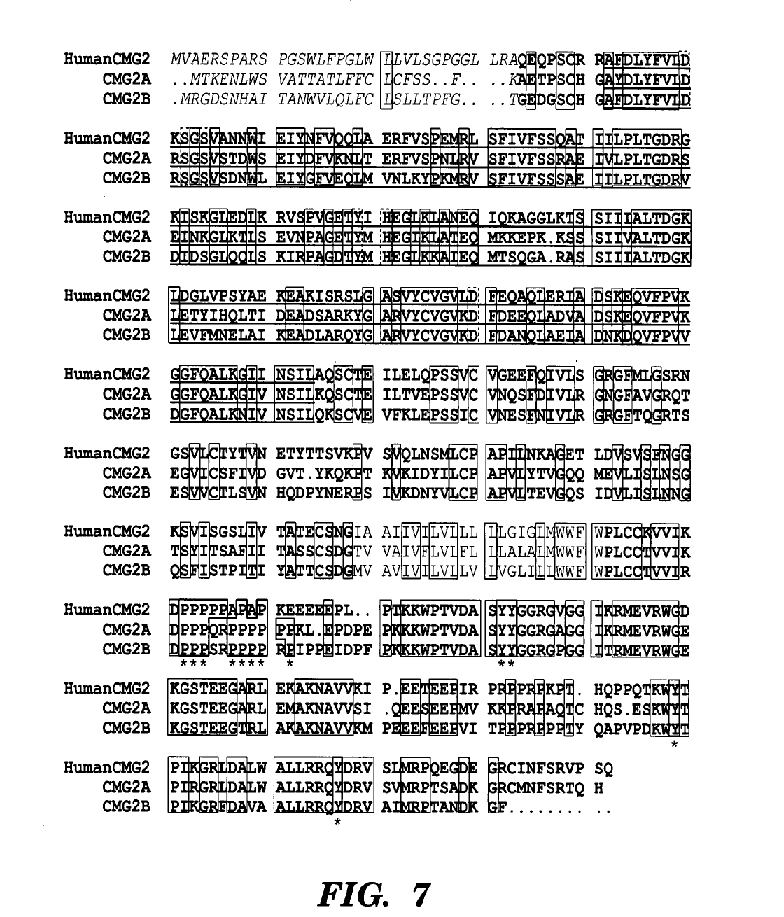

After confirming that zebrafish have conserved orthologs for the ANTXRs (FIG. 7 and data not shown), LeTx was introduced into the circulation of embryos at 48 hpf. The transparency of the zebrafish embryo permitted direct visualization of toxin-induced phenotypes. Pericardial edema was the most conspicuous phenotype, observed in over 90% of injected embryos (n>600; FIG. 2A-D). This effect was specific to LeTx, as single injections of either LF or PA alone did not induce changes in vascular function or in embryo morphology (data not shown).

To examine the progression of cardiovascular changes leading to edema, we monitored vascular effects in real-time over the course of 20 h, using a transgenic line where endothelial cells are labeled with GFP (Tg(fli1:EGFP)y1) (Lawson and Weinstein, 2002).

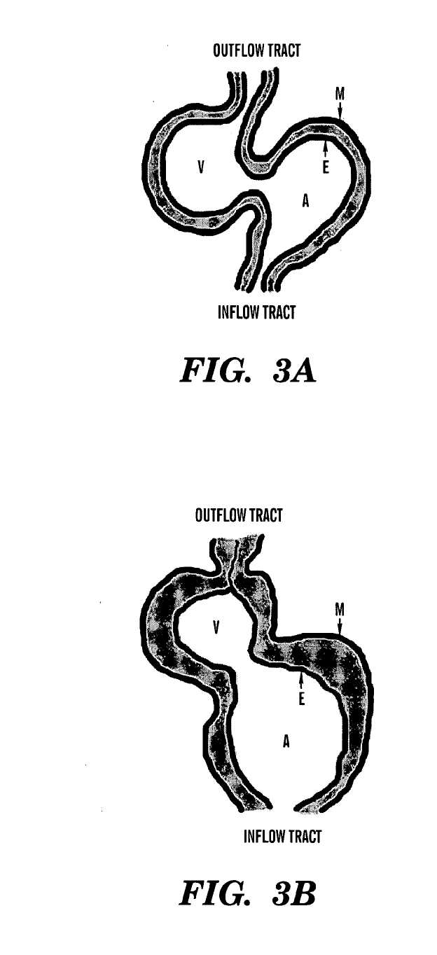

Tg(fli1:EGFP)y embryos were injected with dye alone or dye plus LeTx (75 fMoles LF and 50 fMoles PA), then fluorescent microspheres of 100 nm (blue) and 500 nm (red) were microinjected at the times indicated (data not shown). Control embryos injected with dye alone did not show microsphere leakage (data not shown). Embryos previously injected with LeTx displayed leakage of 100 nm microspheres at the beginning of stage 2 (see below), and 500 nm microspheres at the end of this stage demonstrating extravasated beads into the endocardium (FIG. 3).

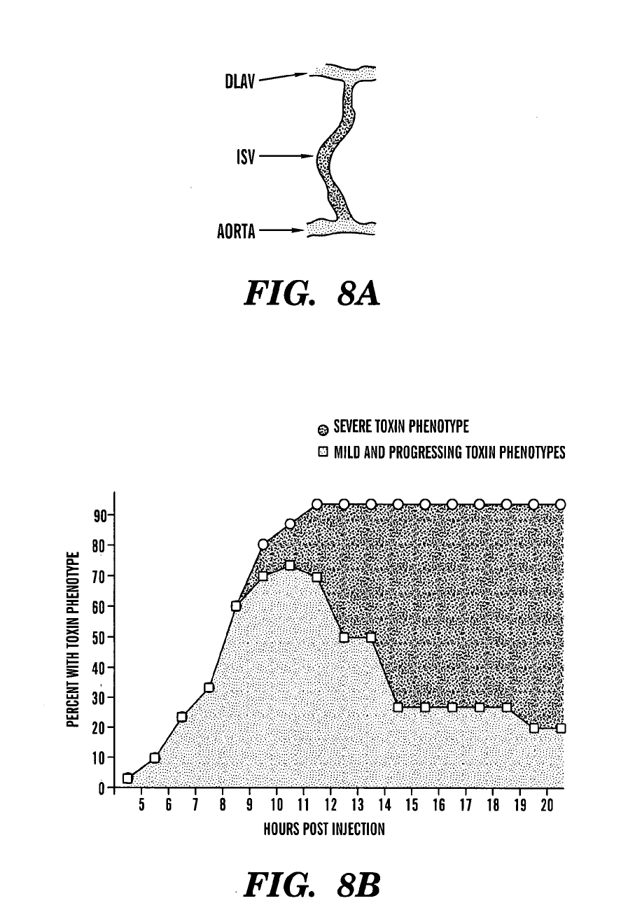

We identified 4 distinct stages of toxin phenotypic progression (FIG. 3 and FIG. 8). LeTx phenotypic effect in the early stage (stage 1) include the beginning of inflow tract regurgitation as compared to a PA control, followed by the progressive collapse of an ISV lumen (stage 2) (FIG. 8A). Each ISV loses its function by the end of this stage. Subsequently the lumen size of the common cardinal vein is highly reduced at the end of stage 3. In stage 4: embryos with a severe phenotype have the outflow tract of the heart completely closed, preventing erythrocytes from exiting the heart from the ventricle. Transgenic zebrafish expressing red fluorescent blood cells Tg(gata1:dsRED) (Traver et al., 2002), were used to visualize this.

Some variation in the onset of toxin effects (starting from 4 to 12 hpi) was observed, but all phenotypes were stabilized by 20 hpi (n>30 embryos; four repeated experiments). We have used the stabilized phenotypes at 20 hpi in subsequent assays to evaluate LeTx effects and its inhibition. To determine that alterations in endothelial permeability were playing an essential role, we also tracked the localization of microinjected fluorescent microspheres at each stage following LeTx exposure. We focused on the heart as fluid accumulation here was consistently observed and the presence of the myocardial layer facilitated trapping of extravasated microspheres through the endothelial layer (data not shown).

The first stage of phenotypic progression lasted about 2 h in which blood cells were seen to accumulate at the inflow tract of the heart with slight blood flow regurgitation and volume increase in both cardiac chambers. Increased permeability was first detected between the endothelial and myocardial layers by the beginning of stage 2 (also lasting about 2 h), as 100 nm microspheres were extravasated (data not shown). By the end of this stage, 500 nm microspheres had leaked into this space in 58% of LeTx injected embryos (n=12). Visually, this could easily be seen as a thickening of the heart wall as fluid accumulated between the 2 layers. In addition, small blood vessels along the trunk of the embryo, termed the intersegmental vessels (ISVs), began to collapse until they could no longer support blood flow. The lumen size of the common cardinal vein, a large vessel that empties into the heart, also started to become progressively narrowed. By stage 3 (about 1 h in length), the outflow tract of the heart was narrowed from ˜20 μm in diameter to 10 μm as measured by blood cells and 500 nm microspheres. This significantly exacerbated the circulatory defect as flow became restricted to the major axial blood vessels. The most distinctive features at the end of this stage were the massive pericardial edema, the pooling of blood at the inflow tract and the toggling of blood cells between the two chambers of the heart.

The final stage of LeTx phenotype development had a duration of about 2 h in which accumulated defects from the previous stages led to an eventual cessation of blood flow in embryos displaying a severe toxin phenotype. An hour into this stage, 75% of embryos displayed microsphere extravasation into the endothelial-myocardial space (500 nm microspheres, n=12). With progressive defects in the outflow and inflow tracts, blood cells became trapped within the heart chambers. In the mild form of the LeTx phenotype, the outflow tract was not completely closed, permitting slow circulation in the axial vessels. The severe and mild toxin phenotypes described here provided distinctive phenotypic classes for visual scoring in subsequent zebrafish assays (see below). By 20 hpi, the end stage time point in our experiments, embryos with severe LeTx effects (93%, n=14) exhibited 500 nm microsphere extravasation in the heart. These microsphere leakage experiments confirmed that endothelial permeability change is an early consequence of LeTx effects. To dissect whether toxin effects utilized conserved host components, a series of validation experiments were performed using reagents to target several points of the mammalian toxin internalization pathway.

Anthrax toxin assembly, internalization and cytosolic delivery are conserved.

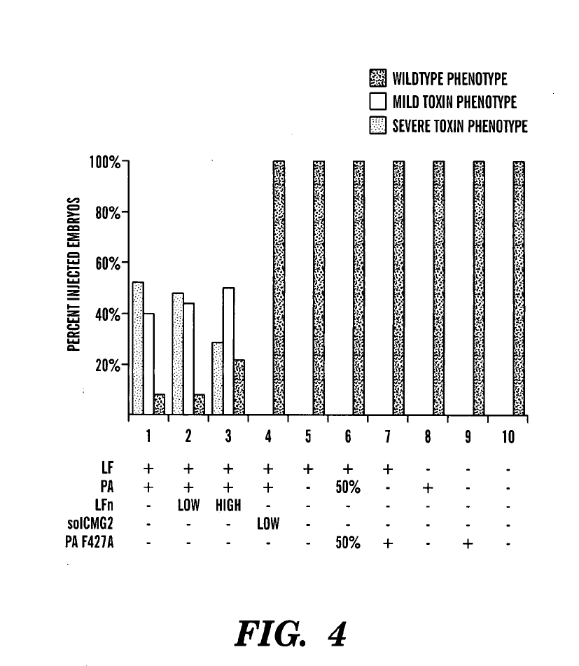

We first focused on PA as an important mediator of host receptor binding and cytosolic delivery of the toxin's enzymatic components. Upon receptor binding and proteolytic cleavage, PA forms a homoheptameric “prepore” that binds LF and the complex is endocytosed. Within the endosome, acidification prompts prepore insertion into the endosomal membrane facilitating the translocation of LF into the cytosol (Collier and Young, 2003; Krantz et al., 2005). We tested several inhibitors against host receptor binding with PA, LF binding to the PA prepore, or translocation of LF. The ability of each inhibitor to alter toxin effects was visually scored for each embryo at 20 hpi as having normal appearance, severe toxin phenotype, or mild toxin phenotype (FIGS. 2 and 4). A sol-CMG2 reagent consisting of the soluble, extracellular region of the human CMG2/ANTXR2 receptor (Scobie et al., 2003, 2005), completely protected against toxin effects, leading to 100% normal embryos (FIG. 4). The isolated N-terminal domain of LF, LFN, provided only mild protection against toxin effects, mirroring its moderate efficacy as an inhibitor in cell culture experiments (Juris et al., submitted). We also used a translocation-defective PA mutant, PA F427A (Krantz et al., 2005), to determine whether toxin steps leading to LF delivery into the cytosol are conserved in the zebrafish. The F427A mutation permits PA to proceed through receptor binding, prepore assembly, internalization and pore formation, but blocks translocation of the LF protein though the pore formed in the endosomal membrane (Krantz et al., 2005). This mutant was inactive in mediating LF action and provided complete protection against the action of LF when mixed 1:1 with wild-type PA, demonstrating the mutant's dominant-negative properties (FIG. 4) (Sellman et al., 2001). These protein-based inhibitors provide strong evidence for the conservation of essential toxin steps in our zebrafish vascular model and demonstrate the use of the fish as a surrogate host for anthrax toxin research.

Anthrax Lethal Toxin Effects are Specific to LF's Enzymatic Activity

Lethal factor is a metalloprotease that cleaves and inactivates MEK kinases (also called MKKs) (Collier and Young, 2003; Duesbery et al., 1998). To determine whether the whole animal effects we observed in the zebrafish model resulted from LF's reported activity, we used a chemical MEK inhibitor, CI-1040 (Allen et al., 2003), to address this question. We observed the greatest effect after treating zebrafish embryos for a 6-hour period, from 48 hpf to 54 hpf, with 2.5 μM of CI-1040, which phenocopied LeTx effects in 100% of the embryos tested (n>30). This chemical phenocopy suggested that LeTx effects were most important during the first 6 h of toxin delivery into the zebrafish vasculature. To further test the specificity of LF action while maintaining all other components of the toxin pathway, we used a fusion protein, LFNDTA, in the zebrafish embryo. Because this fusion protein uses the diphtheria A chain as an enzymatic component (DTA) (Blanke et al., 1996), it should generate a different whole animal outcome compared with LF. LFNDTA and PA produce a different phenotype from LeTx. Zebrafish embryos were injected with PA and LFNDTA or LFNDTA alone at 48 hpi and photographed at 20 hpi as in LeTx experiments. Cycloheximide was used at a concentration of 5 μM for 6 hours to phenocopy the effects of translational inhibition. The combined injection of PA and LFNDTA produced endothelial cell death, a rapid loss of blood vessel function, and widespread necrosis by 5 hpi (data not shown). The development of such drastic effects is not surprising due to the potency of DTA in causing cell death (Yamaizumi et al., 1979). We note that LFNDTA and PA injections generated endothelial cell fragmentation in the intersegmental vessels that was phenocopied by the use of cycloheximide, suggesting that this resulted from the ability of DTA to block translation (data not shown). As with LF injections, these effects were completely blocked by the use of the potent sol-CMG2 inhibitor (data not shown).

Chemical VEGFR Blockade Counteracts LeTx-Induced Vascular Leakage

Although mammalian studies have noted vascular defects toward the final stages of LeTx toxicity (Beall and Dalldorf, 1966; Moayeri et al., 2003; Stearns-Kurosawa et al., 2006), our live imaging of LeTx effects revealed that vascular leakage is an important early consequence of toxicity in our zebrafish model (FIG. 3). As the VEGF-VEGFR signaling pathway is known to be a major regulator of endothelial permeability, we decided to test the ability of chemical VEGFR inhibitors to attenuate LeTx phenotypes. We selected structurally distinct small molecule inhibitors of VEGFR kinase activity: ZM323881 and SU11652. ZM323881, is a highly selective inhibitor of VEGFR2 tyrosine kinase activity (IC50<2 nM) (Whittles et al., 2002). Affinity toward the next closest receptor, VEGFR1, was significantly lower (IC50>50 μM) (Whittles et al., 2002). In human arterial endothelial cells (HAECs), low doses of ZM323881 has been shown to inhibit VEGFR2 activation, but not that of VEGFR1, platelet derived growth factor receptor (PDGFR), epidermal growth factor receptor (EGFR), or hepatocyte growth factor (HGF) (Endo et al., 2003). SU11652 inhibits the activities of the VEGFRs, FGFR, and Kit family members. It has been used successfully to block VEGFR2 activity in C57/CBA mice (Heryanto et al., 2003).

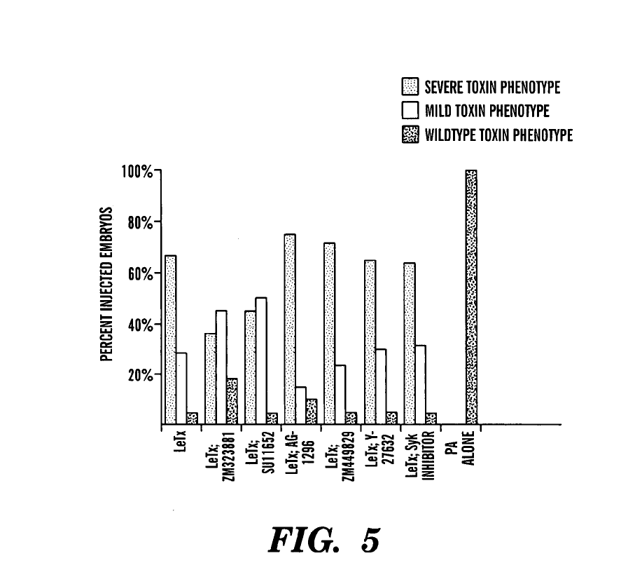

In our zebrafish experiments, 17-30 embryos were used for each inhibitor concentration (FIG. 5 and Table 3). Embryos were pre-incubated with an inhibitor for 30 minutes prior to injection of LeTx. Following injection, each group was maintained at the same concentration of inhibitor for 6 h before the drug was removed by washout. Visual scoring for toxin effects was conducted at 20 hpi. We found that each VEGFR2 inhibitor attenuated LeTx action, as observed by a reduced percentage of embryos having severe toxin effects and an increase in mild or normal phenotypes. Of the two inhibitors, ZM323881, a more VEGFR2-selective inhibitor, provided the best protection against the development of LeTx phenotypes, as indicated by the high percentage of embryos displaying a normal appearance and by reductions in embryos displaying severe and mild LeTx effects (FIG. 5).

To examine the selectivity for VEGFR2 inhibition, we evaluated inhibitors for several other kinase signaling pathways (FIG. 5 and Table 3). Commercially available kinase inhibitors such as AG-1296, ZM449829, Y-27632, and 3-(1-Methyl-1H-indol-3-yl-methylene)-2-oxo-2,3-dihydro-1H-indol-5-sulfonamide were all tested in the same manner. ZM449829 is known to inhibit JAK3, JAK1, EGFR, and STAT5 (Brown et al., 2000; Fraering et al., 2005). AG-1296 is selective for PDGFR and Kit family members (Kovalenko et al., 1997; Krystal et al., 1997; Strutz et al., 2001). Y-27632 is a widely used inhibitor of Rho-associated protein kinases (Uehata et al., 1997). 3-(1-Methyl-1H-indol-3-yl-methylene)-2-oxo-2,3-dihydro-1H-indol-5-sulfonamide is a Syk inhibitor (Lai et al., 2003). None of these kinase inhibitors protected against LeTx effects as observed with VEGFR selective compounds. Collectively, these inhibitor experiments underscore the importance of the endothelial cell as a major target cell type for anthrax toxicity.

TABLE 3
VEGF inhibitors attenuate LeTx action

| | Severe | Mild Toxin | Wildtype | | | |
|---|---|---|---|---|---|---|
| Treatment Condition | Toxin Phenotype | Phenotype | Appearance | n | N | P-value |
| LeTx | 14 | 6 | 1 | 21 | 8 | 1 |
| LeTx; ZM323881 | 8 | 10 | 4 | 22 | 5 | <0.001 |
| LeTx; SU11652 | 10 | 11 | 1 | 22 | 4 | <0.001 |
| LeTx; AG-1296 | 15 | 3 | 2 | 20 | 3 | 0.926 |
| LeTx; ZM449829 | 15 | 5 | 1 | 21 | 3 | 0.571 |
| LeTx; Y-27632 | 13 | 6 | 1 | 20 | 5 | 0.250 |
| LeTx; Syk Inhibitor | 14 | 7 | 1 | 22 | 4 | 0.212 |
| PA Alone | 0 | 0 | 15 | 15 | 8 | <0.001 |

N = Total number of embryos injected.
N = Total number of repeated experiments for each treatment condition (n > 17 for each treatment condition in all experiments).
p-values were obtained using the Chi Square Test and the combined numbers from N.
ZM323881 was added to 1 μM.
SU11652.AG-1296, ZM449829, Y-27632, and the Syk Inhibitor were all added to 1.5 μM.

Attenuation of LeTx Induced Vascular Leakage in Mice

To extend our findings from the zebrafish to mammals, we evaluated the efficacy of VEGFR inhibitors using a well-known vascular permeability test, the Miles assay (Gozes et al., 2006). The Miles assay relies on the leakage of the Evans blue dye (960 Da) as an quantifiable indicator of changes in permeability (Miles and Miles, 1952). As previously described, LeTx induced significant leakage within 60 minutes of intradermal injection in mice (FIG. 6) (Gozes et al., 2006). When combined with LeTx, each of the two VEGFR inhibitors was able to reduce vessel permeability in our experiments (FIG. 6). When the Evans blue dye was extracted from the skin tissue and quantified, we found that these values closely matched the visual results (FIG. 6). Thus, LeTx induced endothelial permeability can be prevented by inhibition of the VEGF signaling pathway in fish and mouse models.

Discussion

Recent clinical reports have placed emphasis on the presence of pleural effusions as a diagnostic indication for human anthrax disease, as it has occurred with high frequency among known cases (nearly 100%) (Kyriacou et al., 2004). During the acute stage of anthrax disease, it is known that high levels of toxins in the bloodstream can lead to death despite the use of antibiotics (e.g. Guarner et al., 2003). LeTx alone can induce death in mammalian models, with concomitant vascular damage and pleural effusions (Beall and Dalldorf, 1966; Moayeri et al., 2003; Ross, 1955; Steams-Kurosawa et al., 2006). Our study has focused on the endothelial effects of LeTx, to provide a mechanistic connection between toxin induced vascular leakage and the role of host endothelial cell signaling pathways. Using our zebrafish model, we uncovered a protective effect against anthrax lethal toxin by blocking the VEGFR signaling pathway. This has been confirmed on intact mammalian blood vessels using the established Miles Assay.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The references cited below and throughout the application are incorporated herein by reference in their entirety.

### REFERENCES

Allen, L. F., Sebolt-Leopold, J., and Meyer, M. B. (2003). CI-1040 (PD184352), a targeted signal transduction inhibitor of MEK (MAPKK). Semin Oncol 30, 105-116.
Avery, R. L., Pieramici, D. J., Rabena, M. D., Castellarin, A. A., Nasir, M. A., and Giust, M. J. (2006). Intravitreal bevacizumab (Avastin) for neovascular age-related macular degeneration. Opthalmology 113, 363-372 e365.
Beall, F. A., and Dalldorf, F. G. (1966). The pathogenesis of the lethal effect of anthrax toxin in the rat. J Infect Dis 116, 377-389.
Beall, F. A., Taylor, M. J., and Thorne, C. B. (1962). Rapid lethal effect in rats of a third component found upon fractionating the toxin of Bacillus anthracis. J Bacteriol 83, 1274-1280.
Blanke, S. R., Milne, J. C., Benson, E. L., and Collier, R. J. (1996). Fused polycationic peptide mediates delivery of diphtheria toxin A chain to the cytosol in the presence of anthrax protective antigen. Proc Natl Acad Sci USA 93, 8437-8442.
Bradley, K. A., Mogridge, J., Mourez, M., Collier, R. J., and Young, J. A. (2001). Identification of the cellular receptor for anthrax toxin. Nature 414, 225-229.
Brown, G. R., Bamford, A. M., Bowyer, J., James, D. S., Rankine, N., Tang, E., Torr, V., and Culbert, E. J. (2000). Naphthyl ketones: a new class of Janus kinase 3 inhibitors. Bioorg Med Chem Lett 10, 575-579.
Chan, J., and Serluca, F. C. (2004). Chemical approaches to angiogenesis. Methods Cell Biol 76, 475-487.
Collier, R. J., and Young, J. A. (2003). Anthrax toxin. Annu Rev Cell Dev Biol 19, 45-70.
Cui, X., Li, Y., Li, X., Laird, M. W., Subramanian, M., Moayeri, M., Leppla, S. H., Fitz, Y., Su, J., Sherer, K., and Eichacker, P. Q. (2007). Bacillus anthracis edema and lethal toxin have different hemodynamic effects but function together to worsen shock and outcome in a rat model. J Infect Dis 195, 572-580.
Duesbery, N. S., Webb, C. P., Leppla, S. H., Gordon, V. M., Klimpel, K. R., Copeland, T. D., Ahn, N. G., Oskarsson, M. K., Fukasawa, K., Paull, K. D., and Vande Woude, G. F. (1998). Proteolytic inactivation of MAP-kinase-kinase by anthrax lethal factor. Science 280, 734-737.
Dvorak, H. F. (2006). Discovery of vascular permeability factor (VPF). Exp Cell Res 312, 522-526.

Endo, A., Fukuhara, S., Masuda, M., Ohmori, T., and Mochizuki, N. (2003). Selective inhibition of vascular endothelial growth factor receptor-2 (VEGFR-2) identifies a central role for VEGFR-2 in human aortic endothelial cell responses to VEGF. J Recept Signal Transduct Res 23, 239-254.
Firoved, A. M., Miller, G. F., Moayeri, M., Kakkar, R., Shen, Y., Wiggins, J. F., McNally, E. M., Tang, W. J., and Leppla, S. H. (2005). Bacillus anthracis edema toxin causes extensive tissue lesions and rapid lethality in mice. Am J Pathol 167, 1309-1320.
Fishman, M. C. Genomics. Zebrafish—the canonical vertebrate. Science 294, 1290-1. (2001).
Fraering, P. C., Ye, W., LaVoie, M. J., Ostaszewski, B. L., Selkoe, D. J., and Wolfe, M. S. (2005). gamma-Secretase substrate selectivity can be modulated directly via interaction with a nucleotide-binding site. J Biol Chem 280, 41987-41996.
Gozes, Y., Moayeri, M., Wiggins, J. F., and Leppla, S. H. (2006). Anthrax lethal toxin induces ketotifen-sensitive intradermal vascular leakage in certain inbred mice. Infect Immun 74, 1266-1272.
Guarner, J., Jernigan, J. A., Shieh, W. J., Tatti, K., Flannagan, L. M., Stephens, D. S., Popovic, T., Ashford, D. A., Perkins, B. A., and Zaki, S. R. (2003). Pathology and pathogenesis of bioterrorism-related inhalational anthrax. Am J Pathol 163, 701-709.
Heryanto, B., Lipson, K. E., and Rogers, P. A. (2003). Effect of angiogenesis inhibitors on oestrogen-mediated endometrial endothelial cell proliferation in the ovariectomized mouse. Reproduction 125, 337-346.
Isogai, S., Horiguchi, M., and Weinstein, B. M. (2001). The vascular anatomy of the developing zebrafish: an atlas of embryonic and early larval development. Dev Biol 230, 278-301.
Jernigan, J. A., Stephens, D. S., Ashford, D. A., Omenaca, C., Topiel, M. S., Galbraith, M., Tapper, M., Fisk, T. L., Zaki, S., Popovic, T., et al. (2001). Bioterrorism-related inhalational anthrax: the first 10 cases reported in the United States. Emerg Infect Dis 7, 933-944.
Juris, J. J, Roman M. A., Bolcome R. E., Chan J., Collier R. J. Crosslinked, multimeric forms of the N-terminal domain of the lethal factor are potent inhibitors of anthrax toxin. Submitted.
Kovalenko, M., Ronnstrand, L., Heldin, C. H., Loubtchenkov, M., Gazit, A., Levitzki, A., and Bohmer, F. D. (1997). Phosphorylation site-specific inhibition of platelet-derived growth factor beta-receptor autophosphorylation by the receptor blocking tyrphostin AG1296. Biochemistry 36, 6260-6269.
Krantz, B. A., Melnyk, R. A., Zhang, S., Juris, S. J., Lacy, D. B., Wu, Z., Finkelstein, A., and Collier, R. J. (2005). A phenylalanine clamp catalyzes protein translocation through the anthrax toxin pore. Science 309, 777-781.
Krystal, G. W., Carlson, P., and Litz, J. (1997). Induction of apoptosis and inhibition of small cell lung cancer growth by the quinoxaline tyrphostins. Cancer Res 57, 2203-2208.
Kyriacou, D. N., Stein, A. C., Yarnold, P. R., Courtney, D. M., Nelson, R. R., Noskin, G. A., Handler, J. A., and Frerichs, R. R. (2004). Clinical predictors of bioterrorism-related inhalational anthrax. Lancet 364, 449-452.
Lai, J. Y., Cox, P. J., Patel, R., Sadiq, S., Aldous, D. J., Thurairatnam, S., Smith, K., Wheeler, D., Jagpal, S., Parveen, S., et al. (2003). Potent small molecule inhibitors of spleen tyrosine kinase (Syk). Bioorg Med Chem Lett 13, 3111-3114.
Lawson, N. D., and Weinstein, B. M. (2002). In vivo imaging of embryonic vascular development using transgenic zebrafish. Dev Biol 248, 307-318.
Leendertz, F. H., Ellerbrok, H., Boesch, C., Couacy-Hymann, E., Matz-Rensing, K., Hakenbeck, R., Bergmann, C., Abaza, P., Junglen, S., Moebius, Y., et al. (2004). Anthrax kills wild chimpanzees in a tropical rainforest. Nature 430, 451-452.
Manley, P. W., Bold, G., Bruggen, J., Fendrich, G., Furet, P., Mestan, J., Schnell, C., Stolz, B., Meyer, T., Meyhack, B., et al. (2004). Advances in the structural biology, design and clinical development of VEGF-R kinase inhibitors for the treatment of angiogenesis. Biochim Biophys Acta 1697, 17-27.
Matsumori, Y., Yano, S., Goto, H., Nakataki, E., Wedge, S. R., Ryan, A. J., and Sone, S. (2006). ZD6474, an inhibitor of vascular endothelial growth factor receptor tyrosine kinase, inhibits growth of experimental lung metastasis and production of malignant pleural effusions in a non-small cell lung cancer model. Oncol Res 16, 15-26.
Miles, A. A., and Miles, E. M. (1952). Vascular reactions to histamine, histamine-liberator and leukotaxine in the skin of guinea-pigs. J Physiol 118, 228-257.
Moayeri, M., Haines, D., Young, H. A., and Leppla, S. H. (2003). Bacillus anthracis lethal toxin induces TNF-alpha-independent hypoxia-mediated toxicity in mice. J Clin Invest 112, 670-682.
Moayeri, M., Martinez, N. W., Wiggins, J., Young, H. A., and Leppla, S. H. (2004). Mouse susceptibility to anthrax lethal toxin is influenced by genetic factors in addition to those controlling macrophage sensitivity. Infect Immun 72, 4439-4447.
Mock, M. & Fouet, A. Anthrax. Annu Rev Microbiol 55, 647-71 (2001).
Mourez, M. Anthrax toxins. Rev Physiol Biochem Pharmacol 152, 135-64 (2004).
Nordberg, B. K., Schmiterloew, C. G., Bergrahm, B., and Lundstroem, H. (1964). Further Pathophysiological Investigations into the Terminal Course of Experimental Anthrax in the Rabbit. Acta Pathol Microbiol Scand 60, 108-116.
Peterson, R. T., and Fishman, M. C. (2004). Discovery and use of small molecules for probing biological processes in zebrafish. Methods Cell Biol 76, 569-591.
Peterson, R. T., Shaw, S. Y., Peterson, T. A., Milan, D. J., Zhong, T. P., Schreiber, S. L., MacRae, C. A., and Fishman, M. C. (2004). Chemical suppression of a genetic mutation in a zebrafish model of aortic coarctation. Nat Biotechnol 22, 595-599.
Pichelmayer, O., Gruenberger, B., Zielinski, C., and Raderer, M. (2006). Bevacizumab is active in malignant effusion. Ann Oncol 17, 1853.
Roberts, J. E., Watters, J. W., Ballard, J. D., and Dietrich, W. F. (1998). Ltx1, a mouse locus that influences the susceptibility of macrophages to cytolysis caused by intoxication with Bacillus anthracis lethal factor, maps to chromosome 11. Mol Microbiol 29, 581-591.
Ross, J. M. (1955). On the histopathology of experimental anthrax in the guinea-pig. Br J Exp Pathol 36, 336-339.
Sandler, A., Gray, R., Perry, M. C., Brahmer, J., Schiller, J. H., Dowlati, A., Lilenbaum, R., and Johnson, D. H. (2006). Paclitaxel-carboplatin alone or with bevacizumab for non-small-cell lung cancer. N Engl J Med 355, 2542-2550.
Santelli, E., Bankston, L. A., Leppla, S. H., and Liddington, R. C. (2004). Crystal structure of a complex between anthrax toxin and its host cell receptor. Nature 430, 905-908.
Schultz, J., Milpetz, F., Bork, P., and Ponting, C. P. (1998). SMART, a simple modular architecture research tool: identification of signaling domains. Proc Natl Acad Sci USA 95, 5857-5864.
Scobie, H. M., Rainey, G. J., Bradley, K. A., and Young, J. A. (2003). Human capillary morphogenesis protein 2 functions as an anthrax toxin receptor. Proc Natl Acad Sci USA 100, 5170-5174.
Scobie, H. M., Thomas, D., Marlett, J. M., Destito, G., Wigelsworth, D. J., Collier, R. J., Young, J. A., and Manchester, M. (2005). A soluble receptor decoy protects rats against anthrax lethal toxin challenge. J Infect Dis 192, 1047-1051.
Sellman, B. R., Nassi, S., and Collier, R. J. (2001). Point mutations in anthrax protective antigen that block translocation. J Biol Chem 276, 8371-8376.
Senger, D. R., Galli, S. J., Dvorak, A. M., Perruzzi, C. A., Harvey, V. S., and Dvorak, H. F. (1983). Tumor cells secrete a vascular permeability factor that promotes accumulation of ascites fluid. Science 219, 983-985.
Stearns-Kurosawa, D. J., Lupu, F., Taylor, F. B., Jr., Kinasewitz, G., and Kurosawa, S. (2006). Sepsis and pathophysiology of anthrax in a nonhuman primate model. Am J Pathol 169, 433-444.
Stern, H. M., Murphey, R. D., Shepard, J. L., Amatruda, J. F., Straub, C. T., Pfaff, K. L., Weber, G., Tallarico, J. A., King, R. W., and Zon, L. I. (2005). Small molecules that delay S phase suppress a zebrafish bmyb mutant. Nat Chem Biol 1, 366-370.
Strutz, F., Zeisberg, M., Renziehausen, A., Raschke, B., Becker, V., van Kooten, C., and Muller, G. (2001). TGF-beta 1 induces proliferation in human renal fibroblasts via induction of basic fibroblast growth factor (FGF-2). Kidney Int 59, 579-592.
Swartz, M. N. Recognition and management of anthrax—an update. N Engl J Med 345, 1621-6 (2001).
Uehata, M., Ishizaki, T., Satoh, H., Ono, T., Kawahara, T., Morishita, T., Tamakawa, H., Yamagami, K., Inui, J., Maekawa, M., and Narumiya, S. (1997). Calcium sensitization of smooth muscle mediated by a Rho-associated protein kinase in hypertension. Nature 389, 990-994.
Weinstein, B. M., Stemple, D. L., Driever, W., and Fishman, M. C. (1995). Gridlock, a localized heritable vascular patterning defect in the zebrafish. Nat Med 1, 1143-1147.
Whittles, C. E., Pocock, T. M., Wedge, S. R., Kendrew, J., Hennequin, L. F., Harper, S. J., and Bates, D. O. (2002). ZM323881, a novel inhibitor of vascular endothelial growth factor-receptor-2 tyrosine kinase activity. Microcirculation 9, 513-522.
Wigelsworth, D. J., Krantz, B. A., Christensen, K. A., Lacy, D. B., Juris, S. J., and Collier, R. J. (2004). Binding stoichiometry and kinetics of the interaction of a human anthrax toxin receptor, CMG2, with protective antigen. J Biol Chem 279, 23349-23356.
Willett, C. G., Boucher, Y., di Tomaso, E., Duda, D. G., Munn, L. L., Tong, R. T., Chung, D. C., Sahani, D. V., Kalva, S. P., Kozin, S. V., et al. (2004). Direct evidence that the VEGF-specific antibody bevacizumab has antivascular effects in human rectal cancer. Nat Med 10, 145-147.
Yamaizumi, M., Uchida, T., Mekada, E., and Okada, Y. (1979). Antibodies introduced into living cells by red cell ghosts are functionally stable in the cytoplasm of the cells. Cell 18, 1009-1014.
Yano, S., Herbst, R. S., Shinohara, H., Knighton, B., Bucana, C. D., Killion, J. J., Wood, J., and Fidler, I. J. (2000). Treatment for malignant pleural effusion of human lung adenocarcinoma by inhibition of vascular endothelial growth factor receptor tyrosine kinase phosphorylation. Clin Cancer Res 6, 957-965.
Zon, L. I. & Peterson, R. T. In vivo drug discovery in the zebrafish. Nat Rev Drug Discov 4, 35-44 (2005).

1. A method for treating anthrax disease in a subject in need thereof, comprising: administering an effective amount of a VEGF inhibitor and a pharmaceutically acceptable carrier.

2. The method of claim 1, wherein the VEGF inhibitor is selected from the group consisting of bevacizumab, VEGF Trap, CP-547,632, AG13736, AG28262, SU5416, SU11248, SU6668, ZD-6474, ZD4190, CEP-7055, PKC 412, AEE788, AZD-2171, sorafenib, vatalanib, pegaptanib octasodium, IM862, DC101, angiozyme, Sirna-027, caplostatin, and neovastat.

3. The method of claim 1, wherein the VEGF inhibitor is administered by pulmonary administration.

4. The method of claim 1, wherein the VEGF inhibitor is administered by parenteral administration.

5. The method of claim 1, wherein the VEGF inhibitor is administered by oral administration.

6. The method of claim 1, further comprising administering antibiotics, wherein the antibiotics are fluoroquinolones, tetracyclines and/or penicillins.

7. The method of claim 1, further comprising administering anthrax anti-toxin agents.

8-21. (canceled)
